(19) United States
(12) Patent Application Publication
(10) Pub. No.: US 2026/0224204 A1
(43) Pub. Date: Aug. 6, 2026

(54) ULTRASOUND DIAGNOSIS SUPPORT APPARATUS AND ULTRASOUND DIAGNOSIS SUPPORT PROGRAM

(71) Applicant: FUJIFILM Corporation, Tokyo (JP)

(72) Inventor: Wataru TOKUDOME, Tokyo (JP)

(73) Assignee: FUJIFILM Corporation, Tokyo (JP)

(21) Appl. No.: 19/437,359

(22) Filed: Dec. 31, 2025

(30) Foreign Application Priority Data
Feb. 4, 2025 (JP) 2025-017030

(51) Int. Cl. A61B 8/00; A61B 8/08; A61B 8/14; G06T 7/62

(52) U.S. Cl. CPC A61B 8/5261; A61B 8/085; A61B 8/0891; A61B 8/14; A61B 8/4245; A61B 8/466; A61B 8/5223; G06T 7/62; G06T 2207/10072; G06T 2207/10132; G06T 2207/30096; G06T 2207/30101

(57) ABSTRACT

A medical image formation unit forms a medical three-dimensional image MI representing an inside of a subject based on medical volume data. A position/posture detection unit detects a position and a posture of an ultrasound probe. A position/posture information conversion unit converts position/posture information indicating the position and the posture of the ultrasound probe defined in a camera coordinate system or a real space coordinate system, which is detected by the position/posture detection unit, into position/posture information in a data coordinate system that is a coordinate system of the medical volume data. A display control unit displays an ultrasound tomographic image USI and a medical three-dimensional image MI on a display, and displays an irradiation region indicator, which indicates an irradiation region of ultrasound waves in the medical three-dimensional image MI, on the medical three-dimensional image MI based on conversion position/posture information.

### CROSS-REFERENCE TO RELATED APPLICATION

This application claims the priority benefit of Japan application serial no. 2025-017030, filed on February 4, 2025. The entirety of the above-mentioned patent application is hereby incorporated by reference herein and made a part of this specification.

### BACKGROUND OF THE INVENTION

### 1. Field of the Invention

The present specification discloses improvements in an ultrasound diagnosis support apparatus and an ultrasound diagnosis support program.

### 2. Description of the Related Art

In the related art, there has been known an ultrasound diagnostic apparatus that forms an ultrasound tomographic image representing a cross section of a subject in an ultrasound scanning plane based on reflected waves from the subject in a case where an ultrasound beam is scanned in the ultrasound scanning plane of the subject. As described above, since the ultrasound diagnostic apparatus can form an image representing an inside of the subject, the ultrasound diagnostic apparatus is used as a medical apparatus.

In addition to the ultrasound diagnostic apparatus, various medical apparatuses that can form an image representing the inside of the subject are known. For example, as the medical apparatus, there is a computed tomography (CT) apparatus that forms a two-dimensional or three-dimensional medical image representing the inside of the subject based on an attenuation amount of X-rays in a case where the subject is irradiated with the X-rays, or a magnetic resonance imaging (MRI) apparatus that forms a two-dimensional or three-dimensional medical image representing the inside of the subject based on electromagnetic waves generated from hydrogen atoms in the subject in a case where the subject is placed in a strong magnetic field and is irradiated with electromagnetic waves from the outside.

In the related art, it has been proposed to support an operator who performs ultrasound diagnosis based on the ultrasound tomographic image formed by the ultrasound diagnostic apparatus and the medical image formed by the medical apparatus other than the ultrasound diagnostic apparatus. For example, WO2004/098414A and JP2008-246264A disclose an ultrasound diagnostic apparatus that forms an ultrasound tomographic image by transmitting and receiving ultrasound waves from an ultrasound probe to the subject, calculates a scan plane (scanning plane) coordinate from a detection signal of a position sensor provided in the ultrasound probe, acquires a reference image by reconstructing volume image data of the subject acquired by a CT apparatus or an MRI apparatus at the same cross section as the ultrasound tomographic image based on the scan plane coordinate, and displays the ultrasound tomographic image and the reference image side by side.

### SUMMARY OF THE INVENTION

Meanwhile, since the ultrasound tomographic image formed by the ultrasound diagnostic apparatus is a cross section of the subject, it may be difficult for the operator to understand which part of the subject is represented by the ultrasound tomographic image currently being displayed. Of course, in a case where the operator operates the ultrasound probe that transmits and receives the ultrasound waves, the operator can naturally understand a position or a posture of the ultrasound probe in contact with a body surface of the subject. However, in the subject, various tissues such as organs and blood vessels may be complexly disposed, and in such a case, it may be difficult for the operator to understand which part of which tissue is represented by the ultrasound tomographic image currently being displayed.

An object of the ultrasound diagnosis support apparatus disclosed in the present specification is to enable the operator to easily understand which part of the subject is the cross section displayed in the ultrasound tomographic image.

An ultrasound diagnosis support apparatus disclosed in the present specification comprises a processor, in which the processor is configured to: acquire medical volume data representing an inside of a subject, which is acquired by a medical apparatus other than an ultrasound diagnostic apparatus; acquire position/posture information indicating a position and a posture of an ultrasound probe that transmits and receives ultrasound waves to and from the subject in an ultrasound scanning plane; convert the position/posture information into conversion position/posture information in a data coordinate system that is a coordinate system of the medical volume data; display an ultrasound tomographic image formed based on a reception signal obtained by the ultrasound probe transmitting and receiving the ultrasound waves to and from the subject, and a medical three-dimensional image representing the inside of the subject, which is formed based on the medical volume data, on a display unit; and display an irradiation region indicator indicating an irradiation region of the ultrasound waves from the ultrasound probe in the medical three-dimensional image on the display unit based on the conversion position/posture information.

The medical volume data may include a tubular tissue inside the subject, and the medical three-dimensional image may represent the tubular tissue.

The processor may be configured to: specify a virtual scanning plane that is the ultrasound scanning plane in the data coordinate system based on the conversion position/posture information; and display, as the irradiation region indicator, a scanning plane image indicating the virtual scanning plane on the medical three-dimensional image.

The processor may be configured to: specify a virtual scanning plane that is the ultrasound scanning plane in the data coordinate system based on the conversion position/posture information; specify, among regions occupied by the tubular tissue in the medical volume data, a region through which the virtual scanning plane passes as a scanned region; and display the scanned region in an identifiable manner on the medical three-dimensional image.

The processor may be configured to: execute detection processing for detecting the tubular tissue from the ultrasound tomographic image; and specify, among regions occupied by the tubular tissue in the medical volume data, a region through which the virtual scanning plane passes and in which the tubular tissue is detected by the detection processing for an ultrasound tomographic image corresponding to the virtual scanning plane as the scanned region.

The medical volume data may be associated with information indicating a target region that is a region in which the ultrasound waves are to be transmitted and received, and the processor may be configured to display a target region indicator indicating a position of the target region in the medical three-dimensional image on the display unit.

The processor may be configured to: specify a virtual scanning plane that is the ultrasound scanning plane in the data coordinate system based on the conversion position/posture information; and execute at least one of output of a notification to an operator or storage processing of the ultrasound tomographic image based on a positional relationship between the virtual scanning plane and the target region.

The target region may be a lesion region, and the processor may be configured to notify the operator in a case where a distance between the virtual scanning plane and the lesion region is equal to or less than a distance threshold value.

The medical volume data may be associated with information indicating a measurement result for each part of the tubular tissue, and the processor may be configured to: specify a virtual scanning plane that is the ultrasound scanning plane in the data coordinate system based on the conversion position/posture information; and display the measurement result for a part of the tubular tissue through which the virtual scanning plane passes on the display unit.

The measurement result may include information indicating a diameter of the tubular tissue, and the processor may be configured to: detect a diameter of the tubular tissue included in the ultrasound tomographic image; and notify an operator in a case where a difference between the diameter of the tubular tissue detected from the ultrasound tomographic image and a diameter of the part of the tubular tissue through which the virtual scanning plane passes, which is associated with the medical volume data, is equal to or more than a difference threshold value.

The processor may be configured to: calculate a parameter related to the tubular tissue based on a measurement value related to the tubular tissue included in the ultrasound tomographic image, which is acquired by performing measurement using the ultrasound tomographic image, and the measurement result for the part of the tubular tissue through which the virtual scanning plane passes, which is associated with the medical volume data; and display the calculated parameter on the display unit.

An ultrasound diagnosis support program disclosed in the present specification is a program causing a computer to execute: acquiring medical volume data representing an inside of a subject, which is acquired by a medical apparatus other than an ultrasound diagnostic apparatus; acquiring position/posture information indicating a position and a posture of an ultrasound probe that transmits and receives ultrasound waves to and from the subject in an ultrasound scanning plane; converting the position/posture information into conversion position/posture information in a data coordinate system that is a coordinate system of the medical volume data; displaying an ultrasound tomographic image formed based on a reception signal obtained by the ultrasound probe transmitting and receiving the ultrasound waves to and from the subject, and a medical three-dimensional image representing the inside of the subject, which is formed based on the medical volume data, on a display unit; and displaying an irradiation region indicator indicating an irradiation region of the ultrasound waves from the ultrasound probe in the medical three-dimensional image on the display unit based on the conversion position/posture information.

According to the ultrasound diagnosis support apparatus disclosed in the present specification, the operator can easily understand which part of the subject is the cross section displayed in the ultrasound tomographic image.

### BRIEF DESCRIPTION OF THE DRAWINGS

FIG. 1 is a schematic diagram showing a configuration of an ultrasound diagnostic system according to the present embodiment.

FIG. 2 is a schematic diagram showing a configuration of an ultrasound diagnostic apparatus according to the present embodiment.

FIG. 3 is a diagram showing a state in which an ultrasound probe is in contact with a lower limb of a subject.

FIG. 4 is a diagram showing a screen displayed on a display in a case of converting the position/posture information into the conversion position/posture information.

FIG. 5 is a diagram showing a display example of a probe image as the irradiation region indicator.

FIG. 6 is a diagram showing a display example of a scanning plane image as the irradiation region indicator.

FIG. 7 is a diagram showing a display example of a scanned region.

FIG. 8 is a diagram showing a first display example of a target region marker as the target region indicator.

FIG. 9 is a diagram showing a second display example of a target region marker as the target region indicator.

FIG. 10 is a diagram showing a display example of a measurement result for a part of a lower limb blood vessel through which the virtual scanning plane passes.

FIG. 11 is a diagram showing a notification example in a case where a difference between a diameter of a tubular tissue detected from an ultrasound tomographic image and a diameter of a part of a tubular tissue through which a virtual scanning plane passes, which is associated with medical volume data, is equal to or more than a difference threshold value.

### DESCRIPTION OF THE PREFERRED EMBODIMENTS

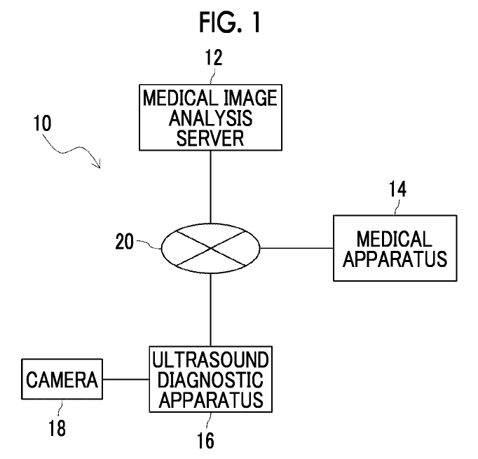

FIG. 1 is a schematic diagram of a configuration of an ultrasound diagnosis support system 10 according to the present embodiment. The ultrasound diagnosis support system 10 includes a medical image analysis server 12, a medical apparatus 14, an ultrasound diagnostic apparatus 16 as an ultrasound diagnosis support apparatus, and a camera 18. The medical image analysis server 12, the medical apparatus 14, and the ultrasound diagnostic apparatus 16 are connected to each other via a communication line 20, such as a wide area network (WAN) or a local area network (LAN), to be communicable with each other. In addition, the ultrasound diagnostic apparatus 16 and the camera 18 are communicably connected to each other by wired or wireless communication.

The medical image analysis server 12 is configured by, for example, a server computer comprising a processor, a memory, a communication interface, and the like. The medical image analysis server 12 is an apparatus that analyzes various medical image data formed by various modalities, such as medical volume data formed by the medical apparatus 14, an ultrasound tomographic image or ultrasound volume data formed by the ultrasound diagnostic apparatus 16.

The medical apparatus 14 is configured by, for example, a CT apparatus or an MRI apparatus. In the present embodiment, the medical apparatus 14 is an apparatus other than the ultrasound diagnostic apparatus. The medical apparatus 14 is an apparatus that forms medical volume data representing an inside of the subject. In particular, in the present embodiment, the medical apparatus 14 forms medical volume data including a tubular tissue inside the subject. In the present embodiment, the tubular tissue is a blood vessel (particularly, a lower limb blood vessel), but the tubular tissue is not limited to the blood vessel, and may be, for example, a lymphatic vessel.

The medical volume data is transmitted to the ultrasound diagnostic apparatus 16 before the ultrasound tomographic image of the subject is formed by the ultrasound diagnostic apparatus 16. The medical volume data may be transmitted from the medical apparatus 14 to the medical image analysis server 12, may be transmitted from the medical image analysis server 12 to the ultrasound diagnostic apparatus 16, or may be directly transmitted from the medical apparatus 14 to the ultrasound diagnostic apparatus 16.

In the present embodiment, the ultrasound diagnosis support system 10 supports an operator who performs an examination of a lower limb blood vessel of the subject (in other words, forms an ultrasound tomographic image representing the lower limb blood vessel of the subject). An example of a flow of the examination of the lower limb blood vessel of the subject is as follows. First, a subject who has subjective symptoms such as swelling or pain in a leg, chest pain, shortness of breath, and palpitation visits a hospital. In a case where a doctor in the hospital determines that the examination of the lower limb blood vessel is necessary for the subject (for example, in a case where the doctor determines that the subject is suspected of having pulmonary embolism), the medical apparatus 14 forms medical volume data of the lower limb of the subject. In addition, the operator forms the ultrasound tomographic image representing the lower limb blood vessel of the subject by using the ultrasound diagnostic apparatus 16. The doctor performs the examination or diagnosis of the subject by using the medical volume data and the ultrasound tomographic image formed as described above. In the present embodiment, the operator who forms the ultrasound tomographic image representing the lower limb blood vessel of the subject by using the ultrasound diagnostic apparatus 16 is supported. The above-described content is merely an example of the examination or diagnosis using the ultrasound diagnosis support system 10, and the examination or diagnosis performed by the ultrasound diagnosis support system 10 is not limited to the above-described content.

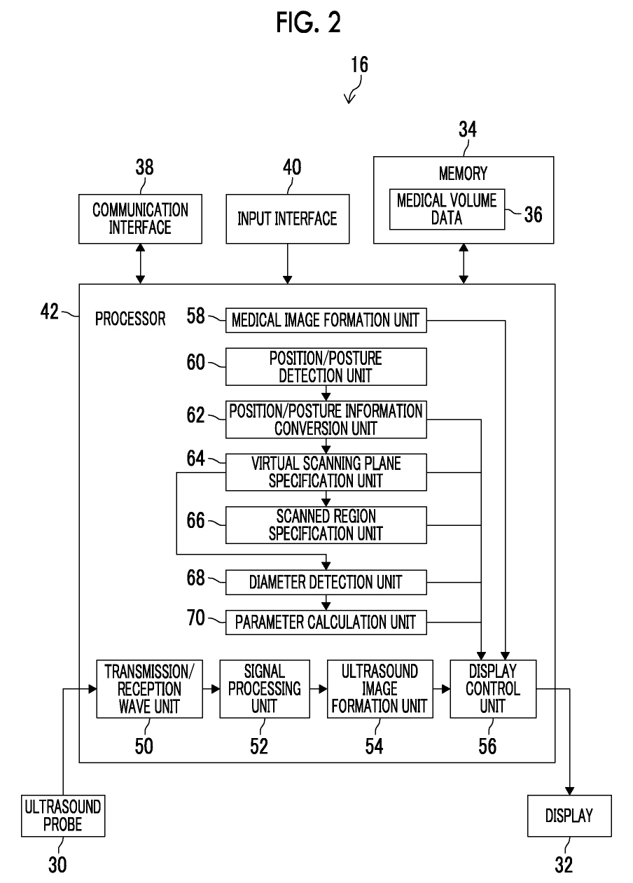

FIG. 2 is a schematic diagram showing a configuration of the ultrasound diagnostic apparatus 16. The ultrasound diagnostic apparatus 16 is a medical apparatus installed in medical institutions, such as a hospital.

An ultrasound probe 30 is a device that transmits and receives ultrasound waves to and from the subject. In particular, in the present embodiment, the ultrasound probe 30 transmits and receives the ultrasound waves to and from the lower limb blood vessel of the subject. The ultrasound probe 30 includes a vibration element array consisting of a plurality of vibration elements that scan the subject with an ultrasound beam. In the present embodiment, the ultrasound probe 30 is a one-dimensional (1D) array probe having a vibration element array consisting of a plurality of vibration elements arranged in one row. Therefore, by supplying the transmission signal from a transmission/reception unit 50 described below to each vibration element, each vibration element generates the ultrasound beam that scans the ultrasound scanning plane.

In the present embodiment, since the ultrasound probe 30 is the 1D array probe, the ultrasound probe 30 transmits and receives the ultrasound waves to and from the subject in the ultrasound scanning plane that is a plane parallel to an arrangement direction of the vibration element array. A size (width and depth, which are a length in a scanning direction of the ultrasound beam) of the ultrasound scanning plane is determined according to known information such as a structure of the ultrasound probe 30 and a setting of the ultrasound diagnostic apparatus 16. For example, the width (scan width) of the ultrasound scanning plane is determined according to the number of vibration elements included in the ultrasound probe 30. In addition, the depth (penetration depth) of the ultrasound scanning plane is determined according to a transmission intensity of the ultrasound beam (that is, an intensity of the transmission signal provided from the transmission/reception unit 50 to the ultrasound probe 30) and the like. In addition, a position and a posture (orientation) of the ultrasound scanning plane are determined based on the position and the posture of the ultrasound probe 30. That is, since the size of the ultrasound scanning plane is determined by known information included in the ultrasound diagnostic apparatus 16, the ultrasound scanning plane is determined in a case where the position and the posture of the ultrasound probe 30 are known.

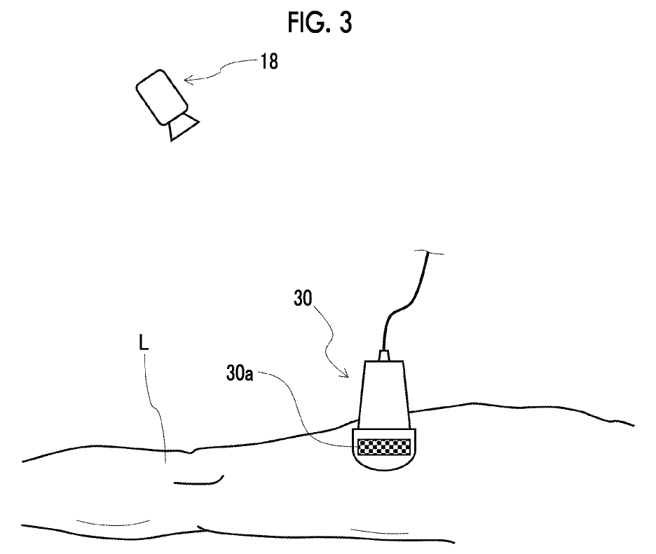

In the present embodiment, the ultrasound probe 30 is provided with a probe detection mark 30a (see FIG. 3). The probe detection mark 30a will be described below.

The display 32 as a display unit is a display device configured by, for example, a liquid crystal display or an organic electroluminescence (EL).

A memory 34 includes a hard disk drive (HDD), a solid state drive (SSD), an embedded multi media card (eMMC), a read only memory (ROM), a random access memory (RAM), or the like. An ultrasound diagnosis support program is stored in the memory 34 in order to operate each unit of the ultrasound diagnostic apparatus 16. The ultrasound diagnosis support program can also be stored in, for example, a computer-readable non-transitory storage medium such as a universal serial bus (USB) memory or a CD-ROM. The ultrasound diagnostic apparatus 16 can read the ultrasound diagnosis support program from such a storage medium and execute the ultrasound diagnosis support program.

In addition, as shown in FIG. 2, medical volume data 36 formed by the medical apparatus 14 is stored in the memory 34.

A communication interface 38 is configured by, for example, a network adapter. The communication interface 38 exhibits a function of communicating with other devices (particularly, the medical apparatus 14, the medical image analysis server 12, and the camera 18) via the communication line 20.

The communication interface 38 receives the medical volume data 36 from the medical image analysis server 12 or the medical apparatus 14. As a result, the ultrasound diagnostic apparatus 16 acquires the medical volume data 36 and stores the medical volume data 36 in the memory 34. The ultrasound diagnostic apparatus 16 may acquire the medical volume data 36 by a method other than the method in which the communication interface 38 receives the medical volume data 36.

As described above, the medical volume data 36 is data representing the inside of the subject. The medical volume data 36 is data in which voxels are arranged three-dimensionally. The medical volume data 36 includes position information of each voxel. In the present embodiment, the position of each voxel constituting the medical volume data 36 is represented in the data coordinate system that is the coordinate system of the medical volume data 36.

In addition, the medical volume data 36 includes information indicating which tissue of the subject each voxel corresponds to (which tissue is represented by each voxel). As a result, it is possible to identify a region occupied by each tissue (for example, a hepatic tissue or the like) of the subject in the medical volume data 36.

In addition, it is preferable that the medical volume data 36 is associated with information indicating a target region that is a region in which the ultrasound waves are to be transmitted and received (in other words, a region in which the ultrasound tomographic image is to be displayed). The target region may be, for example, a bifurcation position of a blood vessel. Alternatively, the target region may be a lesion position. The lesion is, for example, a thrombus. The information indicating the target region may be, for example, a flag assigned to a voxel corresponding to the target region. The information indicating the target region may be automatically assigned to the medical volume data 36 by automatically detecting the bifurcation position of the blood vessel or the lesion position, or may be manually assigned by an operator of the medical apparatus 14 or the like.

Further, it is preferable that the medical volume data 36 is associated with information indicating a measurement result for each tissue of the subject included in the medical volume data 36. In particular, in a case where the medical volume data 36 includes the tubular tissue of the subject as in the present embodiment, it is preferable that the medical volume data 36 is associated with information indicating a measurement result for each part of the tubular tissue. For example, the medical image analysis server 12 or the medical apparatus 14 may be able to execute various measurements by analyzing the medical volume data 36 formed by the medical apparatus 14. Such a measurement result is assigned to the medical volume data 36. The measurement result assigned to the medical volume data 36 is not limited to these, but includes, for example, a measurement result related to the tubular tissue of the subject (for example, a blood vessel diameter or percent stenosis of blood vessel), a measurement result related to the lesion (for example, a thrombus size), and the like. In particular, it is preferable that the position of the medical volume data 36 and the measurement result are associated with each other. For example, the measurement result of the blood vessel diameter may be associated with a blood vessel position at which the blood vessel diameter is measured.

An input interface 40 is configured by, for example, a button, a trackball, or a touch panel. The input interface 40 is used to input an instruction of an operator who uses the ultrasound diagnostic apparatus 16 to the ultrasound diagnostic apparatus 16.

The processor 42 is configured to include, for example, a central processing unit (CPU). The processor 42 exhibits functions of each unit shown in FIG. 2 by the ultrasound diagnosis support program stored in the memory 34.

The transmission/reception unit 50 transmits the transmission signal to the ultrasound probe 30 (specifically, each vibration element of the vibration element array). As a result, the ultrasound probe 30 scans the subject with the ultrasound beam in the ultrasound scanning plane. In addition, the transmission/reception unit 50 receives the reception signal from each vibration element that has received the reflected waves of the ultrasound beam from the subject. The transmission/reception unit 50 includes an adder and a plurality of delay devices corresponding to the respective vibration elements and performs phase alignment and addition processing of aligning and adding phases of the reception signals from the vibration elements using the adder and the plurality of delay devices. As a result, a reception beam signal is formed in which information indicating the signal intensity of reflected waves from the subject is arranged in a depth direction of the subject.

A signal processing unit 52 performs various types of signal processing including filter processing of applying a bandpass filter, wave detection processing, and the like, on the reception beam signal from the transmission/reception unit 50.

An ultrasound image formation unit 54 forms the ultrasound tomographic image (B-mode image) representing the cross section (particularly, an ultrasound transmission/reception surface) of the subject based on the reception beam signal on which the signal processing has been performed by the signal processing unit 52.

The display controller 56 performs control of displaying the ultrasound tomographic image formed by the ultrasound image formation unit 54 on a display 32. In addition, the display control unit 56 performs control of displaying various types of information corresponding to an instruction of each unit of the processor 42, which will be described below, on the display 32.

The medical image formation unit 58 forms a medical three-dimensional image representing the inside of the subject based on the medical volume data 36. Since a method of forming the medical three-dimensional image based on the medical volume data 36 can use a known method, detailed description thereof will be omitted here, but the medical image formation unit 58 forms the medical three-dimensional image by applying a technique such as volume rendering or surface rendering to the medical volume data 36.

As described above, in the present embodiment, since the medical volume data 36 includes the tubular tissue inside the subject, the medical three-dimensional image represents the tubular tissue.

The medical three-dimensional image is not a cross-sectional image reconstructed by cutting out the medical volume data 36 in a certain plane, but is an image representing a state inside the subject (particularly, disposition of the tubular tissue in the present embodiment) in an overview. In other words, in a case of forming the medical three-dimensional image, the medical image formation unit 58 sets a viewpoint of the rendering to a position where the inside of the subject can be viewed in an overview. Since the medical three-dimensional image is displayed on the display 32 as described below, the medical three-dimensional image is naturally a two-dimensional image (in which pixels having signals such as a brightness value and a color value are two-dimensionally arranged), but is an image in which a three-dimensional structure in the medical volume data 36 (three-dimensional space) is represented.

The position/posture detection unit 60 detects the position and the posture of the ultrasound probe 30 to acquire the position/posture information indicating the position and the posture of the ultrasound probe 30. In the present embodiment, the position/posture detection unit 60 detects the position and the posture of the ultrasound probe 30 based on the captured image formed by the camera 18 imaging the ultrasound probe 30. The position and the posture of the ultrasound probe 30 may always fluctuate due to the operation of the operator, but the position/posture detection unit 60 continuously detects the position and the posture of the ultrasound probe 30.

FIG. 3 is a diagram showing a state in which the ultrasound probe 30 is in contact with the lower limb L of the subject. As shown in FIG. 1 or FIG. 3, the ultrasound diagnosis support system 10 includes the camera 18. The camera 18 is configured to include a processor, a communication interface, and the like, in addition to a lens and an image sensor. The camera 18 images the probe detection mark 30a (see FIG. 3) attached to the ultrasound probe 30. The probe detection mark 30a is a mark for detecting the position and the posture of the ultrasound probe 30. Examples of the probe detection mark 30a include an augmented reality (AR) marker.

The camera 18 images the probe detection mark 30a to acquire the captured image and transmits the captured image to the ultrasound diagnostic apparatus 16. The position/posture detection unit 60 detects the position and the posture of the ultrasound probe 30 in the camera coordinate system of the camera 18 by analyzing an image of the probe detection mark 30a shown in the received captured image. The camera coordinate system is a coordinate system in which a position of the camera 18 is an origin, and is, for example, a coordinate system defined by three axes in which a direction of an optical axis of the lens of the camera 18 is set as a z-axis, a direction perpendicular to the z-axis is set as an x-axis, and a direction perpendicular to the x-axis and the z-axis is set as a y-axis. Since a known method can be used as a method of detecting the position and the posture of the ultrasound probe 30 in the camera coordinate system from the image of the probe detection mark 30a included in the captured image, detailed descriptions thereof will be omitted here. The position of the ultrasound probe 30 may be represented by, for example, coordinates of a representative point (for example, a position where the probe detection mark 30a is attached) of the ultrasound probe 30, and the posture of the ultrasound probe 30 may be represented by, for example, a rotation angle about each of the three axes of the camera coordinate system.

The position and the posture of the ultrasound probe 30 may be detected by a method other than the method of analyzing the captured image formed by the camera 18 imaging the probe detection mark 30a. For example, a position/posture sensor such as a magnetic sensor or an acceleration sensor may be provided in the ultrasound probe 30, and the position/posture detection unit 60 may acquire the position/posture information indicating the position and the posture of the ultrasound probe 30 from the position/posture sensor. In this case, the position and the posture of the ultrasound probe 30 are represented by coordinates in a real space coordinate system based on an origin determined by calibration of the sensor.

The position/posture information conversion unit 62 converts the position/posture information indicating the position and the posture of the ultrasound probe 30 defined in the camera coordinate system or the real space coordinate system into the position/posture information in the data coordinate system that is the coordinate system of the medical volume data 36. In the present specification, the position/posture information of the ultrasound probe 30 converted into the data coordinate system is referred to as "conversion position/posture information". An example of the conversion method of the conversion position/posture information is as follows.

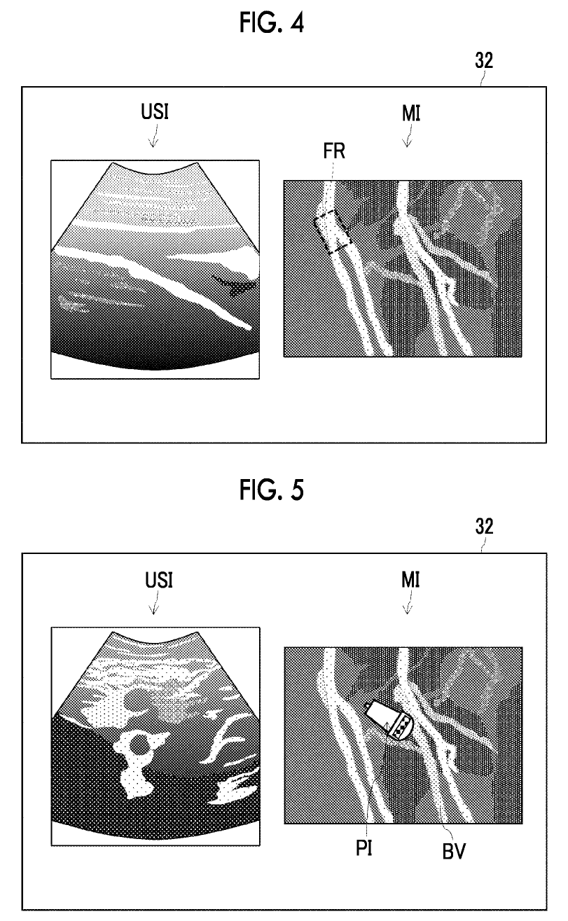

FIG. 4 is a diagram showing a screen displayed on the display 32 in a case of converting the position/posture information into the conversion position/posture information. As shown in FIG. 4, the display control unit 56 displays a real-time ultrasound tomographic image USI (that is, an ultrasound tomographic image formed by transmitting and receiving the ultrasound waves to and from the subject by the ultrasound probe 30 at the current position and posture) formed by the ultrasound image formation unit 54 and a medical three-dimensional image MI formed by the medical image formation unit 58 on the display 32. In order to facilitate the comparison of the two images by the operator, the display control unit 56 may display the ultrasound tomographic image USI and the medical three-dimensional image MI side by side.

Here, a reference position and posture for converting the position/posture information into the conversion position/posture information (in other words, for obtaining a correspondence relationship between the camera coordinate system or the real space coordinate system and the data coordinate system) is determined in advance, and the medical volume data 36 is associated with information indicating the reference position and posture. That is, the reference position and posture are defined in coordinates of the data coordinate system. The reference position and posture is preferably a characteristic part of the subject, and may be, for example, a position and posture in which the ultrasound waves are emitted toward a bifurcation point of the blood vessel. The reference position and posture may be set by the operator.

The operator adjusts and fixes the position and the posture of the ultrasound probe 30 to be the reference position and posture. For example, in a case where the reference position and posture is a position and posture in which the ultrasound waves are emitted toward the bifurcation point of the blood vessel, as shown in FIG. 4, the position and the posture of the ultrasound probe 30 are fixed such that the ultrasound tomographic image USI representing the long-axis cross-sectional image indicating the bifurcation point of the blood vessel is displayed. Here, in order to support the operator, a support image FR indicating the reference position and posture may be displayed in the medical three-dimensional image MI. In addition, a body surface model of the subject may be displayed, and an image indicating the position and the posture of the ultrasound probe 30 at the reference position and posture may be displayed on the body surface model on the display 32. The position/posture detection unit 60 holds the position/posture information indicating the position and the posture of the ultrasound probe 30 in a case of being the reference position and posture in the memory 34 in response to the instruction of the operator.

As described above, the reference position and posture is defined in the data coordinate, and the position/posture detection unit 60 detects the position and the posture of the ultrasound probe 30 in the camera coordinate system or the real space coordinate system. Since the position and posture of the ultrasound probe 30 held in the memory 34 is the reference position and posture in the camera coordinate system or the real space coordinate system, a correspondence relationship between the position and posture of the ultrasound probe 30 held in the memory 34 (camera coordinate system or real space coordinate system) and the reference position and posture (data coordinate system) indicates the correspondence relationship between the camera coordinate system or the real space coordinate system and the data coordinate system. In a case where the correspondence relationship between the camera coordinate system or the real space coordinate system and the data coordinate system is obtained, the position and posture (that is, the conversion position/posture information) of the ultrasound probe 30 (and the ultrasound scanning plane) in the data coordinate system can be obtained based on the position and posture of the ultrasound probe 30 (and the ultrasound scanning plane) in the camera coordinate system or the real space coordinate system detected by the position/posture detection unit 60 in the future.

Obtaining the conversion position/posture information means that the position and the posture of the ultrasound probe 30 in the data coordinate system can be specified. In the present specification, the ultrasound probe 30 in the data coordinate system is referred to as a "virtual probe" to distinguish the ultrasound probe 30 from the actual ultrasound probe 30. Further, based on the position and the posture of the virtual probe in the data coordinate system, the position and the posture of the virtual probe on the medical three-dimensional image MI formed by the medical image formation unit 58 can be specified. For example, the medical image formation unit 58 can form a three-dimensional image of the virtual probe in the data coordinate system and perform the rendering processing on the three-dimensional image of the virtual probe and the medical volume data 36 to represent a two-dimensional image of the virtual probe on the medical three-dimensional image MI. The two-dimensional image of the virtual probe indicates the position and the posture of the virtual probe. Naturally, the position of the virtual probe in the data coordinate system corresponds to the position of the ultrasound probe 30 in the real space coordinate system, and the posture of the virtual probe is the same as the posture of the ultrasound probe 30.

The display control unit 56 displays an irradiation region indicator, indicates the irradiation region of the ultrasound waves in the medical three-dimensional image MI, on the medical three-dimensional image MI based on the conversion position/posture information. FIG. 5 is a diagram showing a display example of a probe image PI as the irradiation region indicator. In the present embodiment, the display control unit 56 displays the real-time ultrasound tomographic image USI and the medical three-dimensional image MI on the display 32, and displays, as the irradiation region indicator, the probe image PI on the medical three-dimensional image MI. A position at which the probe image PI is displayed indicates the position of the virtual probe in the data coordinate system. In addition, the probe image PI has a shape that simulates the ultrasound probe 30, and the posture of the probe image PI indicates the posture of the virtual probe.

By displaying the probe image PI on the medical three-dimensional image MI that displays the state inside the subject in an overview, the operator of the ultrasound diagnostic apparatus 16 can easily understand the position and the posture of the virtual probe in the medical three-dimensional image MI (which also indicates the position and the posture of the ultrasound probe 30 in the real coordinate system). In a case where the position and the posture of the ultrasound probe 30 with respect to each tissue inside the subject can be understood, the operator can estimate the irradiation region of the ultrasound waves from the ultrasound probe 30. Therefore, the probe image PI can be the irradiation region indicator indicating the irradiation region of the ultrasound waves from the ultrasound probe 30.

By displaying the irradiation region indicator in the medical three-dimensional image MI, the operator can easily understand which part of the subject is the cross section displayed in the real-time ultrasound tomographic image USI. In particular, even in a case where the tissues are complexly disposed in the subject, the operator can easily understand the position and the posture of the ultrasound probe 30 with respect to the tissues by the irradiation region indicator.

In the present embodiment, the medical three-dimensional image MI represents a lower limb blood vessel BV that is the tubular tissue. The lower limb blood vessel BV has a complex shape in which an artery and a vein are branched or tortuous, but by displaying the irradiation region indicator in the medical three-dimensional image MI, the operator can easily understand which blood vessel is the lower limb blood vessel BV displayed in the real-time ultrasound tomographic image USI.

The virtual scanning plane specification unit 64 specifies the ultrasound scanning plane in the data coordinate system based on the conversion position/posture information obtained by the position/posture information conversion unit 62 converting the position/posture information of the ultrasound probe 30 acquired by the position/posture detection unit 60. In the present specification, the ultrasound scanning plane in the data coordinate system is referred to as a "virtual scanning plane" to distinguish the ultrasound scanning plane from the actual ultrasound scanning plane. As described above, the size of the ultrasound scanning plane is determined by known information such as a structure of the ultrasound probe 30 and a setting of the ultrasound diagnostic apparatus 16, and the position and the posture of the ultrasound scanning plane are determined based on the position and the posture of the ultrasound probe 30. Therefore, the virtual scanning plane specification unit 64 can specify the virtual scanning plane in the data coordinate system based on the known information, the conversion position/posture information, and a scale of the data coordinate system with respect to the real space coordinate system.

The display control unit 56 may display, as the irradiation region indicator, a scanning plane image indicating the virtual scanning plane specified by the virtual scanning plane specification unit 64 on the medical three-dimensional image MI. The scanning plane image SPI more directly indicates the irradiation region of the ultrasound waves from the ultrasound probe 30 than the probe image PI. Therefore, the operator can more easily understand which part of the subject is the cross section displayed in the real-time ultrasound tomographic image USI than in a case where the probe image PI is displayed as the irradiation region indicator.

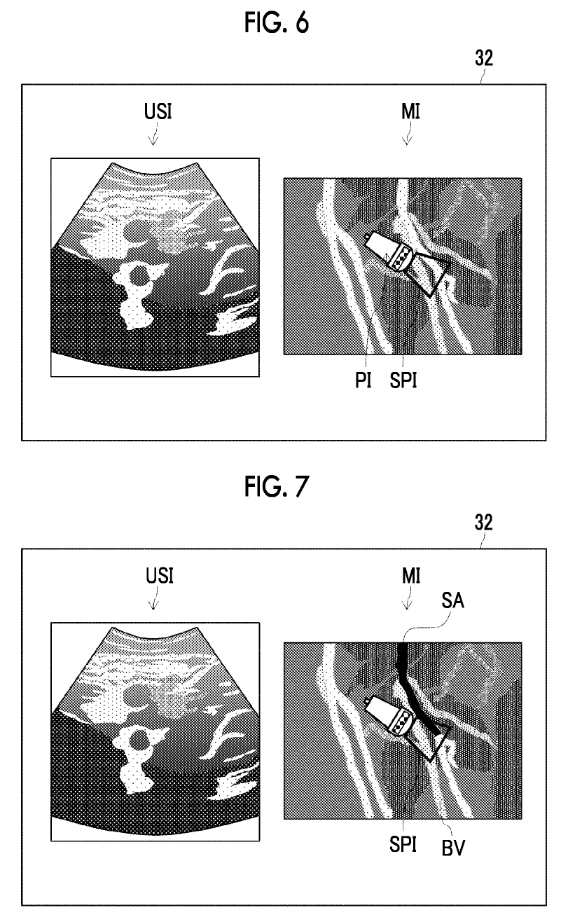

As shown in FIG. 6, both the probe image PI and the scanning plane image SPI may be displayed as the irradiation region indicator.

A process performed by the scanned region specification unit 66 will be described. As described above, the medical volume data 36 includes information indicating which tissue of the subject each voxel corresponds to. Therefore, the scanned region specification unit 66 can specify the region occupied by the tubular tissue in the medical volume data 36 (that is, in the data coordinate system) based on the information.

On the other hand, in a case where the position or the posture of the ultrasound probe 30 is changed by the operator after the correspondence relationship between the camera coordinate system or the real space coordinate system and the data coordinate system is obtained by the position/posture information conversion unit 62, the position or the posture of the virtual scanning plane in the data coordinate system also changes. The virtual scanning plane specification unit 64 can specify a passage path of the virtual scanning plane in the data coordinate system by continuously specifying the virtual scanning plane while the position or the posture of the ultrasound probe 30 changes.

As described above, the region occupied by a hepatic tissue in the data coordinate system (that is, the medical volume data 36) and the passage path of the virtual scanning plane are specified. The scanned region specification unit 66 specifies, among the regions occupied by the tubular tissue in the medical volume data 36, a region through which the virtual scanning plane passes as the scanned region.

The display control unit 56 displays the scanned region specified by the scanned region specification unit 66 in an identifiable manner in the medical three-dimensional image MI. FIG. 7 is a diagram showing a display example of a scanned region SA. The display control unit 56 displays the scanned region SA in the medical three-dimensional image MI in an identifiable manner by, for example, coloring a pixel corresponding to the scanned region SA in the pixel group corresponding to the lower limb blood vessel BV as the tubular tissue in the medical three-dimensional image MI with a predetermined color and not coloring a pixel that does not correspond to the scanned region SA. Of course, the display method of the scanned region SA is not limited thereto.

The scanned region specification unit 66 specifies the scanned region SA in the medical volume data 36, but the scanned region SA in the medical three-dimensional image MI is specified by the rendering processing of the medical image formation unit 58. For example, in the medical volume data 36, a predetermined flag is assigned to the voxel corresponding to the scanned region SA, and among the pixels of the medical three-dimensional image MI obtained by the rendering processing, a pixel corresponding to the voxel to which the flag is assigned can be specified as a pixel constituting the scanned region SA in the medical three-dimensional image MI.

For example, in the examination of the lower limb blood vessel BV, it is examined whether or not a thrombus, a plaque, or the like has occurred in the lower limb blood vessel BV. Although a position in which the thrombus or the plaque is likely to occur is known, the thrombus or the plaque may occur at a position other than the position, so that it is necessary to examine the lower limb blood vessel BV without omission. That is, it is necessary to transmit and receive the ultrasound waves to and from the lower limb blood vessel BV without omission and to form and check the ultrasound tomographic image USI of each part of the lower limb blood vessel BV without omission. As in the present embodiment, by displaying the scanned region SA in the lower limb blood vessel BV, the operator is supported in transmitting and receiving the ultrasound waves to and from the lower limb blood vessel BV without omission.

Here, a case is considered in which the tubular tissue is included in the ultrasound scanning plane due to the ultrasound probe 30 not being appropriately in contact with the subject or an artifact, but the tubular tissue is not appropriately displayed in the ultrasound tomographic image USI. In such a case, it may be inappropriate that the scanned region specification unit 66 simply specifies the region of the tubular tissue through which the virtual scanning plane passes as the scanned region SA. That is, even in a case where the virtual scanning plane formally passes through the tubular tissue, the examination or the check of the tubular tissue cannot be sufficiently performed unless the ultrasound tomographic image USI corresponding to the virtual scanning plane is appropriately displayed.

In consideration of this, the scanned region specification unit 66 may execute the detection processing for detecting the tubular tissue from the ultrasound tomographic image USI by analyzing the real-time ultrasound tomographic image USI. As the detection processing, a known method such as a method using a convolutional neural network (CNN) can be adopted, so that detailed description thereof will be omitted here. Then, the scanned region specification unit 66 may specify, among the regions occupied by the tubular tissue in the medical volume data 36, a region through which the virtual scanning plane passes and in which the tubular tissue is detected by the detection processing for an ultrasound tomographic image USI corresponding to the virtual scanning plane as the scanned region SA. In other words, the scanned region specification unit 66 may not set, among the regions occupied by the tubular tissue in the medical volume data 36, a region through which the virtual scanning plane passes as the scanned region SA in a case where the tubular tissue is not detected by the detection processing.

As a result, a region in which the tubular tissue is appropriately displayed on the ultrasound tomographic image USI can be set as the scanned region SA.

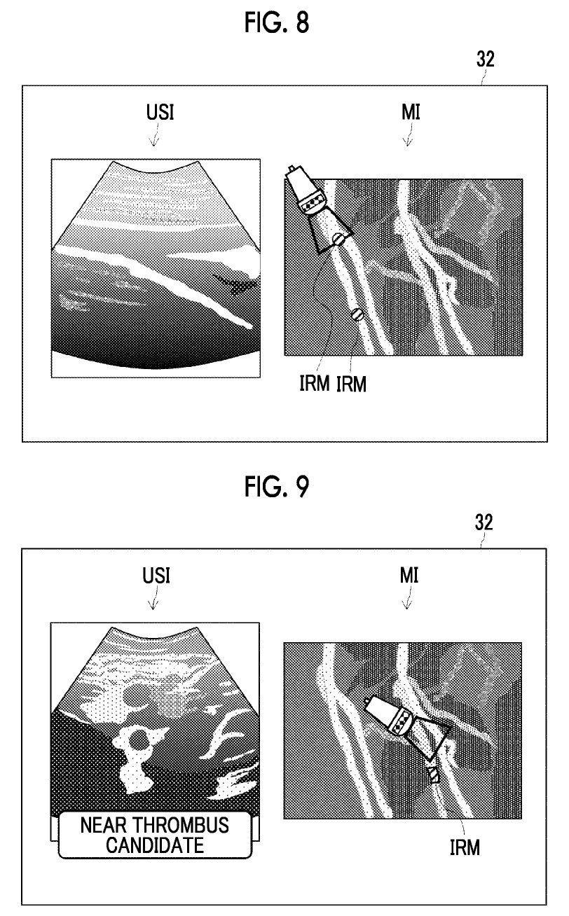

In a case where the medical volume data 36 is associated with information indicating a target region that is a region in which the ultrasound waves are to be transmitted and received, the display control unit 56 may display a target region indicator indicating a position of the target region in the medical three-dimensional image MI on the display 32. FIG. 8 is a diagram showing a first display example of a target region marker IRM as the target region indicator. In particular, FIG. 8 shows a display example of the target region marker IRM in a case where the target region indicating a position in which the lesion region (in the present embodiment, the thrombus or the plaque) is likely to occur is manually assigned to the medical volume data 36. In the present embodiment, the display control unit 56 displays the target region marker IRM on the medical three-dimensional image MI. A position at which the target region marker IRM is displayed indicates the position of the target region in the medical three-dimensional image MI. Of course, the display method of the target region indicator is not limited thereto.

The target region is defined in the medical volume data 36, but the target region in the medical three-dimensional image MI is specified by the rendering processing of the medical image formation unit 58. For example, in the medical volume data 36, a predetermined flag is assigned to the voxel corresponding to the target region, and among the pixels of the medical three-dimensional image MI obtained by the rendering processing, a pixel corresponding to the voxel to which the flag is assigned can be specified as a pixel constituting the target region in the medical three-dimensional image MI.

By displaying the target region indicator, the operator can easily understand the region in which the ultrasound waves are to be transmitted and received.

FIG. 9 is a diagram showing a second display example of the target region marker IRM as the target region indicator. In particular, FIG. 9 shows a display example of the target region indicator in a case where the medical volume data 36 is analyzed in the medical apparatus 14 and the target region indicating a position predicted to be the lesion region is automatically assigned in the medical apparatus 14. In the medical apparatus 14, for example, the position of the lesion region can be predicted from the blood vessel diameter information of the tubular tissue (in the present embodiment, the lower limb blood vessel BV).

In this case, by displaying the target region indicator, the operator can easily understand the region in which the ultrasound waves are to be transmitted and received, and can also easily understand the position predicted to be the lesion region in the medical apparatus 14.

As described above, the target region indicator notifies the operator of the region in which the ultrasound waves are to be transmitted and received, but processing corresponding to the positional relationship between the target region in the medical volume data 36 and the virtual scanning plane specified by the virtual scanning plane specification unit 64 in the data coordinate system may be performed.

For example, the display control unit 56 may output a notification to the operator based on the positional relationship between the virtual scanning plane and the target region. Specifically, the display control unit 56 may output a notification to the operator in a case where a distance between the virtual scanning plane and the target region is equal to or less than a distance threshold value.

FIG. 9 shows a state in which the notification to the operator is displayed in a case where the target region is the lesion region and the distance between the virtual scanning plane and the lesion region is equal to or less than the distance threshold value. As shown in FIG. 9, for example, in a case where the distance between the virtual scanning plane and the lesion region is equal to or less than the distance threshold value, the display control unit 56 displays a message such as "near the thrombus candidate" on the display 32. As a result, the operator can easily understand that the virtual scanning plane, that is, the ultrasound scanning plane is near the lesion region, and can perform the examination of the vicinity of the lesion region in detail.

In the present embodiment, the display control unit 56 displays the message or the like on the display 32 as the notification to the operator, but the notification to the operator may be performed by a sound or light (lamp or the like) instead of or in addition to the display.

In addition, for example, since the target region is a region in which the ultrasound waves are to be transmitted and received, in other words, a region in which the ultrasound tomographic image USI is to be formed, the ultrasound image formation unit 54 may execute the storage processing of the ultrasound tomographic image USI in the memory 34 based on the positional relationship between the virtual scanning plane and the target region. For example, in a case where a freeze instruction to freeze (fix) the ultrasound tomographic image USI displayed on the display 32 is received from the operator, and the distance between the virtual scanning plane and the target region is equal to or less than the distance threshold value, the ultrasound image formation unit 54 may automatically store the ultrasound tomographic image USI in the memory 34. The term "automatically" here means without depending on the instruction of the operator. Alternatively, in a case where the distance between the virtual scanning plane and the target region is equal to or less than the distance threshold value, the ultrasound image formation unit 54 may automatically store the ultrasound tomographic image USI in the memory 34 even in a case where the freeze instruction is not received.

In a case where the medical volume data 36 is associated with information indicating the measurement result for each part of the tubular tissue, the display control unit 56 may display the measurement result on the display 32. In particular, in the present embodiment, the position of the tubular tissue in the medical volume data 36 is associated with the measurement result for the position, and the virtual scanning plane is specified by the virtual scanning plane specification unit 64. Therefore, the measurement result for the part of the tubular tissue through which the virtual scanning plane passes can be specified. Therefore, the display control unit 56 may display the measurement result for the part of the tubular tissue through which the virtual scanning plane passes, among the measurement results associated with the medical volume data 36, on the display 32.

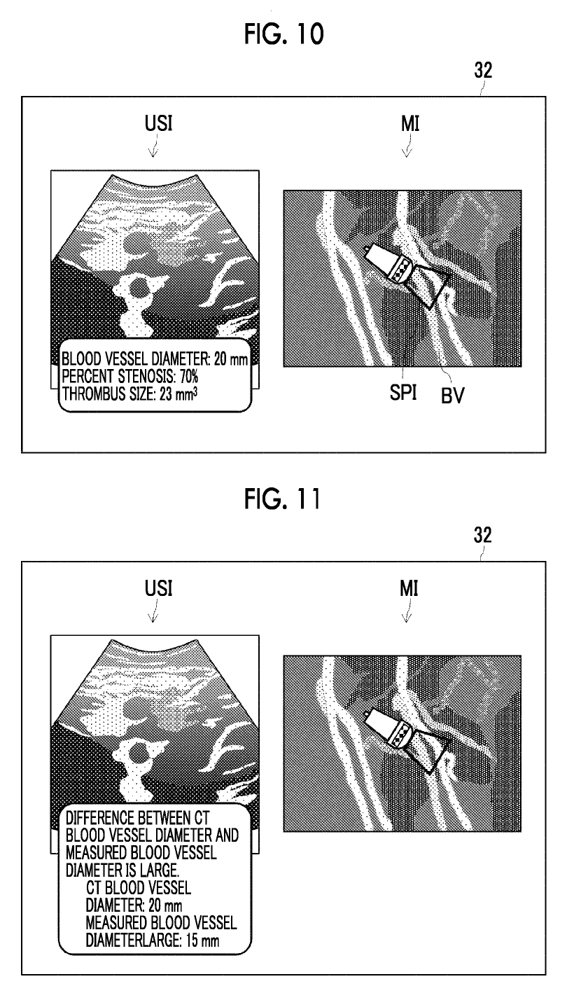

FIG. 10 is a diagram showing a display example of the measurement result for the part of the lower limb blood vessel BV as the tubular tissue through which the virtual scanning plane passes. For example, in a case where the freeze instruction to freeze the ultrasound tomographic image USI is input by the operator, the display control unit 56 displays the measurement result for the part of the lower limb blood vessel BV through which the virtual scanning plane corresponding to the frozen ultrasound tomographic image USI passes. In the example of FIG. 10, a blood vessel diameter, percent stenosis of blood vessel, and a thrombus size are displayed as the measurement result for the part of the lower limb blood vessel BV through which the virtual scanning plane indicated by the scanning plane image SPI passes. Of course, the content of the measurement result is not limited thereto. In a case where the position or the posture of the ultrasound probe 30 is changed by the operator, the position and the posture of the ultrasound scanning plane and the virtual scanning plane are also changed, so that the part of the lower limb blood vessel BV through which the virtual scanning plane passes is also changed. Therefore, the content of the measurement result displayed on the display 32 also dynamically changes in response to the change in the position or the posture of the ultrasound probe 30.

The diameter detection unit 68 detects the diameter of the tubular tissue included in the ultrasound tomographic image USI. For example, the operator freezes the ultrasound tomographic image USI and manually sets a measurement marker to match the diameter of the tubular tissue in the ultrasound tomographic image USI. The diameter detection unit 68 can detect the diameter of the tubular tissue based on the set measurement marker. Alternatively, the diameter detection unit 68 may automatically detect the diameter of the tubular tissue by detecting the tubular tissue from the ultrasound tomographic image USI by a CNN or the like.

As described above, the diameter of the part of the tubular tissue through which the virtual scanning plane passes can be specified among the measurement results associated with the medical volume data 36. On the other hand, the diameter detection unit 68 detects the diameter of the tubular tissue by analyzing the ultrasound tomographic image USI representing the ultrasound scanning plane corresponding to the virtual scanning plane. The diameter obtained from the medical volume data 36 and the diameter obtained from the ultrasound tomographic image USI represent the diameters of the same part of the tubular tissue, so that a difference between the two diameters should not be so large (ideally, the same).

However, in a case where the ultrasound tomographic image USI of the subject is formed in the ultrasound diagnostic apparatus 16 after the medical volume data 36 of the subject is formed, the ultrasound tomographic image USI represents a current state of the subject, while the medical volume data 36 represents a past state of the subject. For example, the blood vessel diameter may change due to a change in a state of the thrombus or the like between the formation of the medical volume data 36 of the subject and the formation of the ultrasound tomographic image USI of the subject in the ultrasound diagnostic apparatus 16. In such a case, the diameter of the tubular tissue detected from the ultrasound tomographic image USI and the diameter of the part of the tubular tissue through which the virtual scanning plane passes, which is associated with the medical volume data 36, may be different from each other.

In addition, in a case where the ultrasound probe 30 is strongly pressed against the body surface of the subject, the tubular tissue (particularly, the vein) may be crushed. Even in such a case, the diameter of the tubular tissue detected from the ultrasound tomographic image USI and the diameter of the part of the tubular tissue through which the virtual scanning plane passes, which is associated with the medical volume data 36, may be different from each other.

Therefore, as shown in FIG. 11, the display control unit 56 may notify the operator in a case where a difference between the diameter of the tubular tissue detected from the ultrasound tomographic image USI and the diameter of the part of the tubular tissue through which the virtual scanning plane passes, which is associated with the medical volume data 36, is equal to or more than a difference threshold value. As a result, the operator can easily understand that the difference between the diameter of the tubular tissue detected from the ultrasound tomographic image USI and the diameter of the part of the tubular tissue through which the virtual scanning plane passes, which is associated with the medical volume data 36, is large.

The parameter calculation unit 70 calculates a parameter related to the tubular tissue based on a measurement value related to the tubular tissue included in the ultrasound tomographic image USI, which is acquired by performing measurement using the ultrasound tomographic image USI, and the measurement result for the part of the tubular tissue through which the virtual scanning plane passes, which is associated with the medical volume data 36. As a result, a comprehensive parameter in which both the measurement value related to the tubular tissue obtained from the ultrasound tomographic image USI and the measurement result for the part of the tubular tissue through which the virtual scanning plane passes, which is associated with the medical volume data 36, are considered can be calculated.

The display control unit 56 displays the parameter calculated by the parameter calculation unit 70 on the display 32.

Although the ultrasound diagnosis support apparatus according to the present disclosure has been described above, the ultrasound diagnosis support apparatus according to the present disclosure is not limited to the above-described embodiment, and various changes can be made without departing from the gist thereof.

For example, in the above-described embodiment, the ultrasound diagnostic apparatus 16 has each of the functions of the medical image formation unit 58, the position/posture detection unit 60, the position/posture information conversion unit 62, the virtual scanning plane specification unit 64, the scanned region specification unit 66, the diameter detection unit 68, and the parameter calculation unit 70, but each of the functions of these units may not necessarily be exhibited by the ultrasound diagnostic apparatus 16. For example, these functions may be exhibited by another device such as the medical image analysis server 12.

Further, all of the above-described functions may not be exhibited by one device, and the above-described functions may be exhibited by cooperation of a plurality of devices.

In the present embodiment, each processing is executed by any computer. In addition, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for specific use, a workstation, or another system capable of executing each processing.

The processor may be configured using one or more pieces of hardware, and the type of hardware is not limited. For example, the processor may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), or a neural processing unit (NPU). The types of hardware may be a combination of different types of hardware. In a case in which the plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor is not limited to the above-described order, and may be changed as appropriate. The hardware is composed of an electric circuit (circuitry) in which circuit elements such as semiconductor elements are combined.

The program may be software such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

The embodiments of the present invention can also be applied to a program and a program product.

What is claimed is:

1. An ultrasound diagnosis support apparatus comprising:
a processor,
wherein the processor is configured to:
acquire medical volume data representing an inside of a subject, which is acquired by a medical apparatus other than an ultrasound diagnostic apparatus;
acquire position/posture information indicating a position and a posture of an ultrasound probe that transmits and receives ultrasound waves to and from the subject in an ultrasound scanning plane;
convert the position/posture information into conversion position/posture information in a data coordinate system that is a coordinate system of the medical volume data;
display an ultrasound tomographic image formed based on a reception signal obtained by the ultrasound probe transmitting and receiving the ultrasound waves to and from the subject, and a medical three-dimensional image representing the inside of the subject, which is formed based on the medical volume data, on a display unit; and
display an irradiation region indicator indicating an irradiation region of the ultrasound waves from the ultrasound probe in the medical three-dimensional image on the display unit based on the conversion position/posture information.

2. The ultrasound diagnosis support apparatus according to claim 1,
wherein the medical volume data includes a tubular tissue inside the subject, and
the medical three-dimensional image represents the tubular tissue.

3. The ultrasound diagnosis support apparatus according to claim 1,
wherein the processor is configured to:
specify a virtual scanning plane that is the ultrasound scanning plane in the data coordinate system based on the conversion position/posture information; and
display, as the irradiation region indicator, a scanning plane image indicating the virtual scanning plane on the medical three-dimensional image.

4. The ultrasound diagnosis support apparatus according to claim 2,
wherein the processor is configured to:
specify a virtual scanning plane that is the ultrasound scanning plane in the data coordinate system based on the conversion position/posture information;
specify, among regions occupied by the tubular tissue in the medical volume data, a region through which the virtual scanning plane passes as a scanned region; and
display the scanned region in an identifiable manner on the medical three-dimensional image.

5. The ultrasound diagnosis support apparatus according to claim 4,
wherein the processor is configured to:
execute detection processing for detecting the tubular tissue from the ultrasound tomographic image; and
specify, among regions occupied by the tubular tissue in the medical volume data, a region through which the virtual scanning plane passes and in which the tubular tissue is detected by the detection processing for an ultrasound tomographic image corresponding to the virtual scanning plane as the scanned region.

6. The ultrasound diagnosis support apparatus according to claim 1,
wherein the medical volume data is associated with information indicating a target region that is a region in which the ultrasound waves are to be transmitted and received, and
the processor is configured to display a target region indicator indicating a position of the target region in the medical three-dimensional image on the display unit.

7. The ultrasound diagnosis support apparatus according to claim 6,
wherein the processor is configured to:
specify a virtual scanning plane that is the ultrasound scanning plane in the data coordinate system based on the conversion position/posture information; and
execute at least one of output of a notification to an operator or storage processing of the ultrasound tomographic image based on a positional relationship between the virtual scanning plane and the target region.

8. The ultrasound diagnosis support apparatus according to claim 7,
wherein the target region is a lesion region, and
the processor is configured to notify the operator in a case where a distance between the virtual scanning plane and the lesion region is equal to or less than a distance threshold value.

9. The ultrasound diagnosis support apparatus according to claim 2,
wherein the medical volume data is associated with information indicating a measurement result for each part of the tubular tissue, and
the processor is configured to:
specify a virtual scanning plane that is the ultrasound scanning plane in the data coordinate system based on the conversion position/posture information; and
display the measurement result for a part of the tubular tissue through which the virtual scanning plane passes on the display unit.

10. The ultrasound diagnosis support apparatus according to claim 9,
wherein the measurement result includes information indicating a diameter of the tubular tissue, and
the processor is configured to:
detect a diameter of the tubular tissue included in the ultrasound tomographic image; and
notify an operator in a case where a difference between the diameter of the tubular tissue detected from the ultrasound tomographic image and a diameter of the part of the tubular tissue through which the virtual scanning plane passes, which is associated with the medical volume data, is equal to or more than a difference threshold value.

11. The ultrasound diagnosis support apparatus according to claim 9,
wherein the processor is configured to:
calculate a parameter related to the tubular tissue based on a measurement value related to the tubular tissue included in the ultrasound tomographic image, which is acquired by performing measurement using the ultrasound tomographic image, and the measurement result for the part of the tubular tissue through which the virtual scanning plane passes, which is associated with the medical volume data; and
display the calculated parameter on the display unit.

12. A non-transitory computer-readable storage medium storing an ultrasound diagnosis support program causing a computer to execute:
acquiring medical volume data representing an inside of a subject, which is acquired by a medical apparatus other than an ultrasound diagnostic apparatus;
acquiring position/posture information indicating a position and a posture of an ultrasound probe that transmits and receives ultrasound waves to and from the subject in an ultrasound scanning plane;
converting the position/posture information into conversion position/posture information in a data coordinate system that is a coordinate system of the medical volume data;
displaying an ultrasound tomographic image formed based on a reception signal obtained by the ultrasound probe transmitting and receiving the ultrasound waves to and from the subject, and a medical three-dimensional image representing the inside of the subject, which is formed based on the medical volume data, on a display unit; and
displaying an irradiation region indicator indicating an irradiation region of the ultrasound waves from the ultrasound probe in the medical three-dimensional image on the display unit based on the conversion position/posture information.
